(19) United States
(12) Patent Application Publication
(10) Pub. No.: US 2015/0289779 A1
(43) Pub. Date: Oct. 15, 2015

(54) SYSTEM AND METHOD FOR DIAGNOSIS OF FOCAL CORTICAL DYSPLASIA

(71) Applicants: Bruce Fischl, Cambridge, MA (US); William A. Copen, Boston, MA (US)

(72) Inventors: Bruce Fischl, Cambridge, MA (US); William A. Copen, Boston, MA (US)

(21) Appl. No.: 14/435,246

(22) PCT Filed: Oct. 2, 2013
(86) PCT No.: PCT/US13/63052

§ 371 Date: Apr. 13, 2015

Related U.S. Application Data
(60) Provisional application No. 61/715,779, filed on Oct. 18, 2012.

(51) Int. Cl. A61B 5/055; G01R 33/385; G06K 9/46; A61B 5/107; A61B 5/00; G06T 7/00; G01R 33/54; G01R 33/34

(52) U.S. Cl. CPC A61B 5/055; G01R 33/543; G01R 33/385; G01R 33/34; A61B 5/1072; A61B 5/4064; A61B 5/0046; A61B 5/0042; A61B 5/0037; G06T 7/0012; G06T 7/0081; G06K 9/4609; A61B 2576/026; G06K 2209/051; G06T 2207/10088; G06T 2207/30016

(57) ABSTRACT

A system and method for automatic detection of potential focal cortical dysplasias through magnetic resonance imaging. The method includes acquiring image data of a subject brain at a first resolution, analyzing the acquired image data to determine a thickness of cerebral gray matter, and matching the left cerebral hemisphere to the right cerebral hemisphere based on corresponding geometric features of the hemispheres. The method also includes generating a difference map comparing corresponding thicknesses of the hemispheres, identifying regions of abnormal differences in thickness as potential regions containing focal cortical dysplasias, and acquiring image data of the regions of abnormal differences in thickness at a second resolution. The method further includes generating images of the regions of abnormal differences in thickness from the acquired image data and displaying the images.

### CROSS-REFERENCE TO RELATED APPLICATIONS

This application is based on, claims priority to, and incorporates herein by reference in its entirety U.S. Provisional Application Ser. No. 61/715,779, filed Oct. 18, 2013, and entitled “SYSTEM AND METHOD FOR DIAGNOSIS OF FOCAL CORTICAL DYSPLASIA USING MAGNETIC RESONANCE IMAGING.”

### STATEMENT REGARDING FEDERALLY SPONSORED RESEARCH

This invention was made with government support under NS052585 and P41-RR14075 awarded by the National Institutes of Health. The government has certain rights in the invention.

### BACKGROUND OF THE INVENTION

The present invention relates generally to systems and methods for medical imaging and, more particularly, the invention relates to systems and methods for automated detection of focal cortical dysplasias in medical images.

Epilepsy, a common neurological disorder characterized by recurrent unprovoked seizures, exacts a large toll upon society in terms of both quality of life and health care costs. The prevalence of epilepsy in the United States has been estimated at approximately 0.68%, suggesting that over two million Americans are currently affected (Hauser et al., 1991). Furthermore, the morbidity of epilepsy is great, in part because epilepsy, unlike many other neurologic disorders, affects patients of all ages and can significantly impair a patient's quality of life for many years. Indeed, the incidence of new cases of epilepsy is seen in the first year of life, thus accounting for the cost-intensive nature of the disorder. One analysis of data collected in 1995 estimated that the lifetime cost of American cases newly diagnosed in that year was $11.1 billion, whereas the annual cost of all cases of epilepsy in the United States at that time was $12.5 billion (Begley et al., 2000).

Malformations of cortical development (“MCD”) constitute the most common cause of seizures in children and the second most frequent cause in adults. One type of malformation that causes epilepsy is Focal Cortical Dysplasia (“FCD”), which is a structural brain lesion that occurs along the surface of the brain and results from abnormal formation of the brain during gestation. FCDs can be classified as Type I if they occur with isolated architectural abnormalities, such as dyslamination, with subtypes depending upon the presence (IB) or absence (IA) of giant or immature neurons; Type II (or “Taylor-type”) if they contain architectural abnormalities and dysmorphic neurons, subtyped contingent on the presence (IIB) or absence (IIA) of balloon cells; or Type III, which are defined to be FCDs associated with another lesion.

Fortunately, surgical resection of FCD lesions provides curative results in approximately 49% to 72% of patients. Surgery is a particularly appealing option for the treatment of FCD because these lesions typically cause medically refractory seizures in young patients, with many years of seizure-impaired life ahead of them, and because early age at the time of surgery does not appear to decrease the likelihood of successful surgery. Furthermore, for the approximately 30% of epilepsy patients whose seizures cannot be controlled by medication, brain surgery is the only remaining therapeutic option.

Focal brain lesions, and in particular FCD, can be identified in magnetic resonance imaging (MRI). For example, FCDs can be diagnosed based on observing characteristics such as increased thickness of the cortical gray matter, blurring of the gray/white junction, abnormal “texture” in cortical gray matter, and/or abnormal signal intensities in either the gray matter, the subjacent white matter or both due to the presence of balloon cell-containing lesions. These are subtle variations in the thickness and signal characteristics in the brain's cerebral cortex, a structure that is so highly convoluted and anatomically irregular that it is difficult for the human eye to detect small abnormalities, thus making FCD often very difficult to detect by even the most experienced subspecialist neuroradiologists.

For example, during visual analysis of MRI images, the foldings of the cortex make diagnosis exceedingly difficult as a visual estimation of the thickness (defined as the distance between the gray/white boundary and the pial surface) will invariably be inaccurate in regions where the surfaces are not parallel to either each other or one of the cardinal imaging planes. Substantially accurate measurements of the thickness of the cortex can be achieved during imaging, but only using an isotropic voxel resolution of 1 millimeter or below. Unfortunately, images acquired at this resolution across the entire brain represent an enormous amount of data for a radiologist to examine in order to detect a subtle abnormality. Furthermore, merely screening for the general location of an abnormality is insufficient. A precise identification of lesion margins on MRI can be critical because complete resection of the lesion is an important predictor of a successful outcome in seizure reduction.

In addition, a form of FCDs called Focal Transmantle Dysplasias (“FTDs”) are subtle abnormalities that, in the majority of cases, are only visible on high resolution MRI images, such as fluid attenuated inversion recovery (“FLAIR”) or T2-weighted scans. As discussed above, high-resolution MRI places a great burden on neuroradiologists as they must scan through hundreds or thousands of slices in order to detect the subtle FLAIR brightening (the hallmark of FTDs) on only a few images. This identification is made even more complex by the trajectory of the thin trail or pathways of abnormal white matter signal in FTDs as it is unlikely to lie completely in any one imaging slice.

Approaches for specifically diagnosing FTDs have looked to previous general approaches for diagnosing FCDs, including detecting absolute cortical thickness as a primary feature together with T1-weighted gray-matter intensity, intensity gradient across the gray/white boundary, as well as gray matter “density” produced by Statistical Parametric Mapping (“SPM”) software. Other general approaches have sought to visually enhance FCDs using T1-weighted images as input. Also, as discussed above, diagnosis through the use of FLAIR signal intensities can significantly increase detection accuracy of FCDs and, in particular, FTDs (as they present most prominently as regions of abnormal FLAIR intensity). While these approaches can exhibit good sensitivity in homogeneous, controlled research studies, they are likely to fail to detect FTDs in practice. More specifically, since small FTDs that are difficult to diagnose frequently present without detectable focal cortical thickening, typical “thickness-detection” approaches for diagnosing cannot be used. Furthermore, analyzing FLAIR images for small changes in signal intensities is a very time-consuming, and thus impractical, approach.

It would therefore be desirable to provide a method and MRI system to automatically detect abnormal cortical thickening, allowing radiologists to focus on a reduced area of regions that may contain FCDs. It would also be desirable to provide a method and system for specifically detecting potential FTDs and identifying their white matter pathways.

### SUMMARY OF THE INVENTION

The present invention overcomes the aforementioned drawbacks by providing a system and method for automatically detecting and localizing focal cortical dysplasias. The invention can be used to accurately register the cerebral hemisphere on one side of the brain to the hemisphere on the other side. The present invention recognizes that corresponding locations have approximately the same thickness, except for regions that have dysplasias. Following identification of these regions, high spatial resolution data is acquired only of these regions so that high resolution images of the regions can be displayed for manual examination. As the output images only include regions of potential dysplasias rather than the whole brain, this invention dramatically limits the amount of data that a neuroradiologist must view in order to make a diagnosis.

The present invention further overcomes the aforementioned drawbacks by providing a system and method for automatically detecting focal transmantle dysplasias. The invention can determine abnormally bright MRI signal intensities from acquired image data, model abnormal migration paths based on these determinations, and derive summary measures from the paths that are predictive of the existence and location of one or more focal transmantle dysplasias.

Thus, in accordance with one aspect of the invention, a magnetic resonance imaging (“MRI”) system includes a magnet system configured to generate a polarizing magnetic field about at least a portion of a subject arranged in the MRI system, a magnetic gradient system including a plurality of magnetic gradient coils configured to apply at least one magnetic gradient field to the polarizing magnetic field, and a radio frequency (“RF”) system configured to apply an RF field to the subject and to receive magnetic resonance signals therefrom in parallel. The MRI system also includes a computer system programmed to control operation of the magnetic gradient system and RF system to acquire image data of a subject brain at a first resolution, analyze the acquired image data to determine a thickness of cerebral gray matter, and match a left cerebral hemisphere to a right cerebral hemisphere based on corresponding geometric features of the left cerebral hemisphere and the right cerebral hemisphere. The computer system is further programmed to generate a difference map comparing corresponding thicknesses of the left cerebral hemisphere and the right cerebral hemisphere, identify regions of abnormal differences in thickness on the difference map as potential regions containing focal cortical dysplasias, control operation of the magnetic gradient system and RF system to acquire image data of the regions of abnormal differences in thickness at a second resolution, generate images of the regions of abnormal differences in thickness from the acquired image data, and display the images.

In accordance with another aspect of the invention, a method for automatic detection of potential focal cortical dysplasias through magnetic resonance imaging includes acquiring image data of a subject brain at a first resolution, analyzing the acquired image data to determine a thickness of cerebral gray matter, and matching a left cerebral hemisphere to a right cerebral hemisphere based on corresponding geometric features of the left cerebral hemisphere and the right cerebral hemisphere. The method also includes generating a difference map comparing corresponding thicknesses of the left cerebral hemisphere and the right cerebral hemisphere, determine regions of abnormal differences in thickness on the difference map as potential regions containing focal cortical dysplasias, and acquiring image data of the regions of abnormal differences in thickness at a second resolution. The method further includes generating images of the regions of abnormal differences in thickness from the acquired image data and displaying the images.

In accordance with yet another aspect of the invention, a system includes a computer system programmed to access image data of a subject brain, analyze the acquired image data to estimate signal intensity distributions of the acquired image data relative to compartments of the subject brain, and determine at least two anchor points of a potential transmantle path. The computer system is further caused to generate an initial transmantle path between the two anchor points and determine a posterior distribution including an optimal transmantle path and additional transmantle paths based on the initial transmantle path. The computer system is further programmed to apply a correction technique to remove cortical geometric effects from the posterior distribution, conclude a corrected optimal transmantle path from the corrected posterior distribution as a focal transmantle dysplasia, and display an image highlighting the focal transmantle dysplasia.

In accordance with yet another aspect of the invention, a method for automatic detection of a focal transmantle dysplasia through magnetic resonance imaging includes acquiring image data of a subject brain, analyzing the acquired image data to determine at least two anchor points of a potential transmantle path, generating an initial transmantle path between the two anchor points, and determining a posterior distribution including an optimal transmantle path and additional transmantle paths based on the initial transmantle path. The method also includes applying a correction technique to remove cortical geometric effects from the posterior distribution, concluding a corrected optimal transmantle path from the corrected posterior distribution as the focal transmantle dysplasia, and displaying an image highlighting the focal transmantle dysplasia.

The foregoing and other aspects and advantages of the invention will appear from the following description. In the description, reference is made to the accompanying drawings which form a part hereof, and in which there is shown by way of illustration a preferred embodiment of the invention. Such embodiment does not necessarily represent the full scope of the invention, however, and reference is made therefore to the claims and herein for interpreting the scope of the invention.

### BRIEF DESCRIPTION OF THE DRAWINGS

FIG. 1 is a block diagram of an example of a magnetic resonance imaging (“MRI”) system for use with the present invention.

FIG. 2 is a flow chart setting forth the steps of an example process for automatic detection of potential focal cortical dysplasias through magnetic resonance imaging in accordance with one aspect of the present invention.

FIG. 3 is an example difference map image generated during the process steps set forth in FIG. 2.

FIG. 4 is a flow chart setting for the steps of an example process for automatic detection of a focal transmantle dysplasia through magnetic resonance imaging in accordance with another aspect of the present invention.

FIGS. 5A-5C are a series of images illustrating T2-SPACE FLAIR image scans from a study incorporating methods of the present invention.

FIG. 6 is another series of images illustrating T2-SPACE FLAIR image scans from the study incorporating methods of the present invention.

FIG. 7 is yet another series of images illustrating inflated cortical surface models from the study incorporating methods of the present invention.

FIG. 8 is a receiver operating characteristic (“ROC”) curve computed based on results from the study incorporating methods of the present invention.

### DETAILED DESCRIPTION OF THE INVENTION

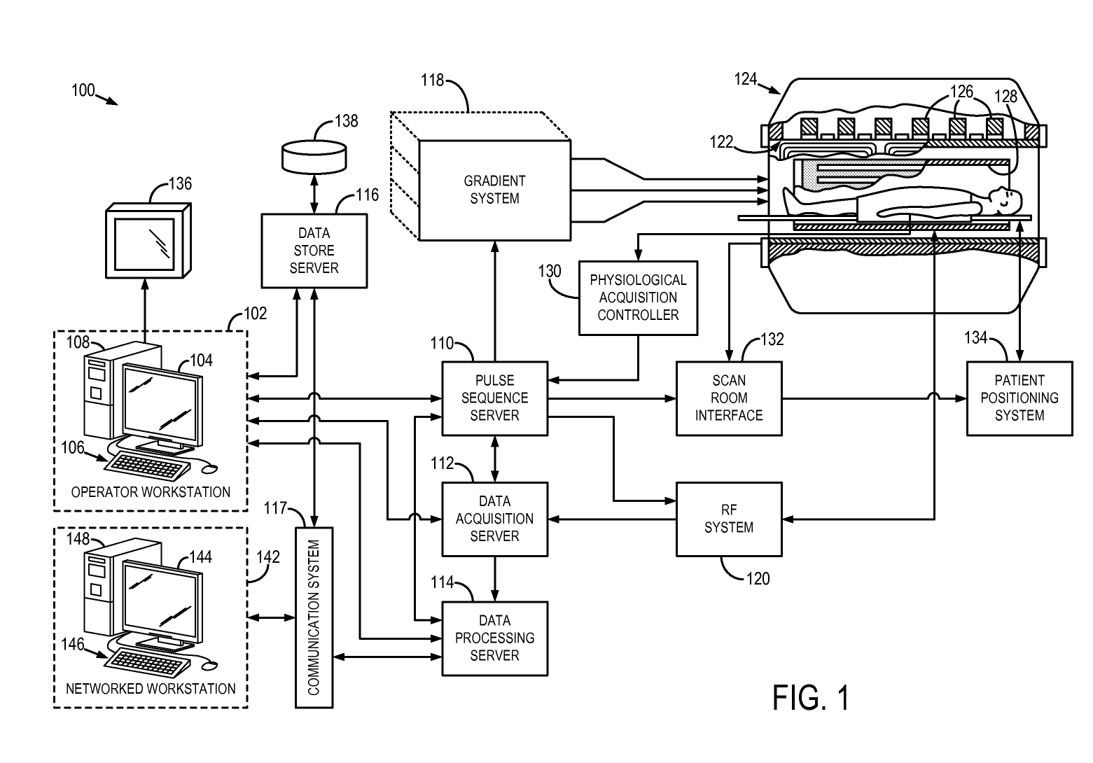

Referring particularly now to FIG. 1, an example of a magnetic resonance imaging (MRI) system 100 is illustrated. The MRI system 100 includes an operator workstation 102, which will typically include a display 104, one or more input devices 106, such as a keyboard and mouse, and a processor 108. The processor 108 may include a commercially available programmable machine running a commercially available operating system. The operator workstation 102 provides the operator interface that enables scan prescriptions to be entered into the MRI system 100. In general, the operator workstation 102 may be coupled to four servers: a pulse sequence server 110; a data acquisition server 112; a data processing server 114; and a data store server 116. The operator workstation 102 and each server 110, 112, 114, and 116 are connected to communicate with each other. For example, the servers 110, 112, 114, and 116 may be connected via a communication system 117, which may include any suitable network connection, whether wired, wireless, or a combination of both. As an example, the communication system 117 may include both proprietary or dedicated networks, as well as open networks, such as the internet.

The pulse sequence server 110 functions in response to instructions downloaded from the operator workstation 102 to operate a gradient system 118 and a radiofrequency (“RF”) system 120. Gradient waveforms necessary to perform the prescribed scan are produced and applied to the gradient system 118, which excites gradient coils in an assembly 122 to produce the magnetic field gradients and used for position encoding magnetic resonance signals. The gradient coil assembly 122 forms part of a magnet assembly 124 that includes a polarizing magnet 126 and a whole-body RF coil 128.

RF waveforms are applied by the RF system 120 to the RF coil 128, or a separate local coil (not shown in FIG. 1), in order to perform the prescribed magnetic resonance pulse sequence. Responsive magnetic resonance signals detected by the RF coil 128, or a separate local coil (not shown in FIG. 1), are received by the RF system 120, where they are amplified, demodulated, filtered, and digitized under direction of commands produced by the pulse sequence server 110. The RF system 120 includes an RF transmitter for producing a wide variety of RF pulses used in MRI pulse sequences. The RF transmitter is responsive to the scan prescription and direction from the pulse sequence server 110 to produce RF pulses of the desired frequency, phase, and pulse amplitude waveform. The generated RF pulses may be applied to the whole-body RF coil 128 or to one or more local coils or coil arrays (not shown in FIG. 1).

The RF system 120 also includes one or more RF receiver channels. Each RF receiver channel includes an RF preamplifier that amplifies the magnetic resonance signal received by the coil 128 to which it is connected, and a detector that detects and digitizes the quadrature components of the received magnetic resonance signal. The magnitude of the received magnetic resonance signal may, therefore, be determined at any sampled point by the square root of the sum of the squares of the and components:

M=\sqrt{I²+Q²} Eqn. (1);

and the phase of the received magnetic resonance signal may also be determined according to the following relationship:

$$ϕ = {tan}^{- 1} ( \frac{Q}{I} ) . \qquad Eqn . ( 2 )$$

The pulse sequence server 110 also optionally receives patient data from a physiological acquisition controller 130. By way of example, the physiological acquisition controller 130 may receive signals from a number of different sensors connected to the patient, such as electrocardiograph (“ECG”) signals from electrodes, or respiratory signals from respiratory bellows or other respiratory monitoring device. Such signals are typically used by the pulse sequence server 110 to synchronize, or “gate,” the performance of the scan with the subject's heart beat or respiration.

The pulse sequence server 110 also connects to a scan room interface circuit 132 that receives signals from various sensors associated with the condition of the patient and the magnet system. It is also through the scan room interface circuit 132 that a patient positioning system 134 receives commands to move the patient to desired positions during the scan.

The digitized magnetic resonance signal samples produced by the RF system 120 are received by the data acquisition server 112. The data acquisition server 112 operates in response to instructions downloaded from the operator workstation 102 to receive the real-time magnetic resonance data and provide buffer storage, such that no data is lost by data overrun. In some scans, the data acquisition server 112 does little more than pass the acquired magnetic resonance data to the data processor server 114. However, in scans that require information derived from acquired magnetic resonance data to control the further performance of the scan, the data acquisition server 112 is programmed to produce such information and convey it to the pulse sequence server 110. For example, during prescans, magnetic resonance data is acquired and used to calibrate the pulse sequence performed by the pulse sequence server 110. As another example, navigator signals may be acquired and used to adjust the operating parameters of the RF system 120 or the gradient system 118, or to control the view order in which k-space is sampled. In still another example, the data acquisition server 112 may also be employed to process magnetic resonance signals used to detect the arrival of a contrast agent in a magnetic resonance angiography (MRA) scan. By way of example, the data acquisition server 112 acquires magnetic resonance data and processes it in real-time to produce information that is used to control the scan.

The data processing server 114 receives magnetic resonance data from the data acquisition server 112 and processes it in accordance with instructions downloaded from the operator workstation 102. Such processing may, for example, include one or more of the following: reconstructing two-dimensional or three-dimensional images by performing a Fourier transformation of raw k-space data; performing other image reconstruction algorithms, such as iterative or backprojection reconstruction algorithms; applying filters to raw k-space data or to reconstructed images; generating functional magnetic resonance images; calculating motion or flow images; and so on.

Images reconstructed by the data processing server 114 are conveyed back to the operator workstation 102 where they are stored. Real-time images are stored in a data base memory cache (not shown in FIG. 1), from which they may be output to operator display 112 or a display 136 that is located near the magnet assembly 124 for use by attending physicians. Batch mode images or selected real time images are stored in a host database on disc storage 138. When such images have been reconstructed and transferred to storage, the data processing server 114 notifies the data store server 116 on the operator workstation 102. The operator workstation 102 may be used by an operator to archive the images, produce films, or send the images via a network to other facilities.

The MRI system 100 may also include one or more networked workstations 142. By way of example, a networked workstation 142 may include a display 144; one or more input devices 146, such as a keyboard and mouse; and a processor 148. The networked workstation 142 may be located within the same facility as the operator workstation 102, or in a different facility, such as a different healthcare institution or clinic.

The networked workstation 142, whether within the same facility or in a different facility as the operator workstation 102, may gain remote access to the data processing server 114 or data store server 116 via the communication system 117. Accordingly, multiple networked workstations 142 may have access to the data processing server 114 and the data store server 116. In this manner, magnetic resonance data, reconstructed images, or other data may exchanged between the data processing server 114 or the data store server 116 and the networked workstations 142, such that the data or images may be remotely processed by a networked workstation 142. This data may be exchanged in any suitable format, such as in accordance with the transmission control protocol (TCP), the internet protocol (IP), or other known or suitable protocols.

As will be described, using an MRI system such as the MRI system 100 described above, one aspect of the present invention provides a method for detecting and localizing focal cortical dysplasias (“FCDs”). Generally, the present invention includes a procedure to determine the thickness of the cortex based on acquired MR data and to accurately register the cerebral hemisphere on one side of the brain to the hemisphere on the other side. Abnormal differences in thickness between corresponding locations on either hemisphere indicate possible regions that have dysplasias. These regions can be identified based on detection of the abnormal thickness differences and instructions can be generated to facilitate the acquisition of high spatial-resolution data in the identified regions.

Higher-resolution images of the entire brain are typically not acquired during scans, as they would represent too much data for a radiologist to realistically examine. The present invention allows the radiologist to only focus on, and acquire additional images for, regions where suspected dysplasias exist based on discrepancies in gray matter thickness. This approach provides a feasible, realistic volume of scans to be examined by the clinician. Generally, the measured thickness of the cortex is dependent on many factors such as age, gender, intracranial volume, and MR sequence used when scanning the subject. Because of these multiple factors, it is difficult to determine whether a given thickness value is unusual with respect to a normal population without matching all these factors, something that is highly impractical to do in practice. However, the present invention allows for a self-contained procedure for detecting abnormally thick cortex by using the left/right symmetry of the subject's own brain. As discussed above, the method detects thickness abnormalities as regions in which one side of the brain is significantly thicker than the other. Although lateralization, varying regional thicknesses, and conventional “whole brain analysis” concepts would tend to lead one away from such a construct, the present invention unexpectedly discovered that, when the left and right hemispheres are appropriately aligned, corresponding locations have approximately the same thickness except for regions that have a dysplasias. Accordingly, the present invention provides a system and method that can use the patient as their own control, thereby reliably matching for demographic and acquisition factors.

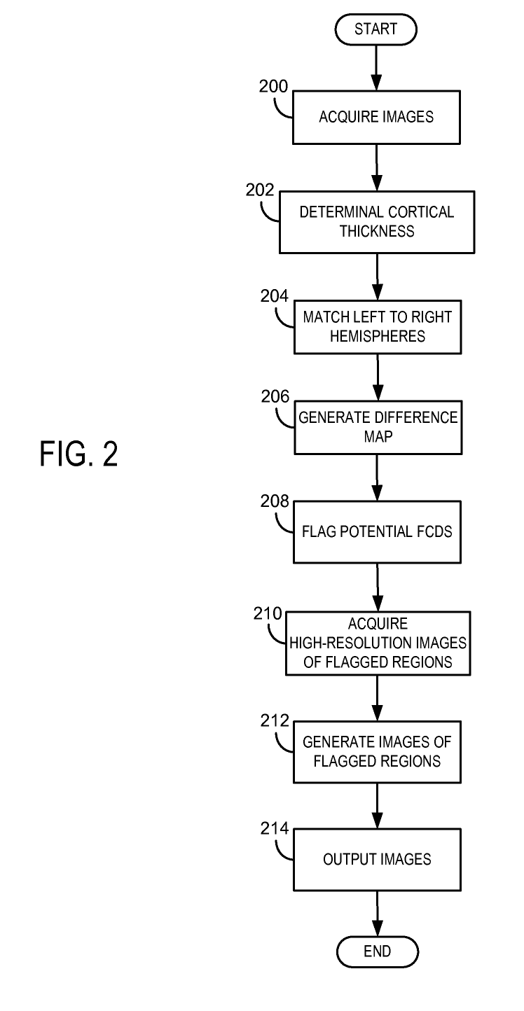

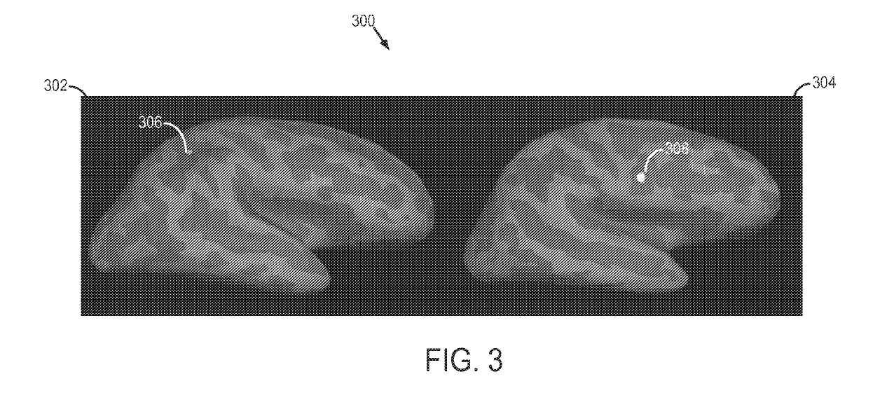

More specifically, an example of a method for detecting and localizing FCDs using an MRI system will be described with respect to FIG. 2. First, images of the cortex are acquired at a first resolution using the MRI system (process block 200). For instance, T1-weighted images can be acquired with the first resolution, such as a 1 millimeter (“mm”) or 1.25 mm isotropic resolution. Thus, the first resolution may be a low or standard resolution image to manage scan time. Next, the images are processed to build models of the bottom and top of the cerebral gray matter (that is, the gray-white boundary and the pial surface) or other processing techniques that can be used to provide a measure of cortical thickness at each point in each hemisphere (process block 202). Following this, the geometries of the cortical hemispheres are used to establish correspondence from one hemisphere to the other so that corresponding geometric features (such as sulci and gyri) are matched across the hemispheres (process block 204). This is desirable because thickness varies over the brain with, for example, frontal regions being thicker than occipital ones, and gyri in general being thicker than sulci. Next, a difference map can be generated by subtracting the thickness at each point in the right hemisphere from the corresponding point of the left hemisphere, or vice versa (process block 206). In the resulting difference map, when subtracting thickness values in the right hemisphere from those the left hemisphere, regions of large positive value can be identified as indicating a potential dysplasia in the left hemisphere, while regions of large negative values indicate a potential dysplasia in the right hemisphere. An example difference map 300 of the left hemisphere 302 and the right hemisphere 304 is illustrated in FIG. 3, showing a potential FCD 306 in the left hemisphere 302 (as would be indicated by a large positive difference value), and a potential FCD 308 in the right hemisphere 304 (as would be indicated by a large negative difference value). For example, a “large” value may be in the range of three or greater millimeters for some patients. However, in some instances, it may be desirable to quantify “large” values differently, for example, to increase or decrease sensitivity and, thereby, draw greater or lesser clinician attention to a variation. For example, depending upon the value of “large” may be selected in coordination with the spatial resolution of the images acquired at the first resolution. Thus, “large” may be quantified using a user-selected or system-selected “threshold,” such as described below.

Specifically, regions of large positive or negative difference values in the generated difference map may be identified or flagged as potential regions including FCDs (process block 208). A threshold difference value, such as about three millimeters, may be set for qualifying measured differences. In such an example, differences of about three millimeters or greater would be identified as abnormally large and flagged as potential regions indicative of dysplasias. However, the clinician may select the actual threshold value to be, for example, less than three millimeters, such as two millimeters, or greater than three millimeters, such as four or five millimeters. Of course, the clinician or system may decide to use fractions of millimeters.

Once the regions are flagged, instructions may be communicated for further data acquisition of these regions (process block 210). More specifically, additional data acquisition can be executed to obtain images having a second spatial resolution that is higher than the spatial resolution of the images obtained in process block 200 (process block 212). For instance the second spatial resolution may be 1 mm or below. Generally, the second spatial resolution may not be isotropic. Rather, the second spatial resolution may generally include a higher in-plane resolution than its through-plane resolution. As one specific example, T1-weighted images can be acquired with a second spatial resolution, such as with a 1 mm through-plane resolution and a 0.5 mm×0.5 mm in-plane resolution. As a more general example, through-plane resolution may be on the order of 1 mm, or more, while in-plane resolution can be below 1 mm.

Images of the flagged regions can be output or displayed (process block 214), which will allow the clinician to automatically receive high resolution images of just the regions in the vicinity of suspected dysplasias for easier, less time-consuming visual analysis. In some cases, the difference map may also be displayed to the clinician. With reference to the MRI system 100, one or more of the above steps may be performed at the data processing server 114 or workstation 102/142 or other suitable server or computer.

Thus, one aspect of the present invention is a diagnostic support utility for detecting and localizing FCDs, which may be self-contained. High-quality neuroimaging data may be input and the output may be a small set of brain regions that may possibly contain a dysplasia, dramatically limiting the amount of data that a neuroradiologist must view in order to make a diagnosis.

According to another aspect of the present invention, a computer-aided diagnosis method to specifically detect FCDs, in particular Focal Transmantle Dysplasias (“FTDs”), in high-resolution MRI is provided. The signature characteristic of FTDs is the existence of abnormally bright T2 or Fluid Attenuated Inversion Recovery (“FLAIR”) MRI intensities extending from the cortex to the ventricles, indicative of the presence of balloon cells in white matter and a failure of cellular differentiation and migration during development. Generally, this aspect of the present invention provides a method to detect these signature characteristics, model abnormal migration paths, and derive summary measures that are predictive of the existence and location of one or more FTDs.

More specifically, models are constructed to specify the start (cortex-side) and end (ventricle-side) points of paths based on Magnetization Prepared Rapid Gradient Echo (“MPRAGE”) or 3D FLAIR images, for example by explicitly finding trails of atypically bright intensity on a FLAIR image. The paths are modeled using low-dimensional splines to enforce smoothness and to reduce the complexity of the estimation of optimal pathways. Probabilistic techniques are used that allow computation of the optimal path for each location in the cortex, as well as all likely, although less optimal, paths that form the useful region of the posterior distribution of path probability (for example, as generated by perturbing the splines). Modeling can be accomplished using software tools such as the FreeSurfer suite of neuroanatomical models (developed by the Laboratory for Computational Neuroimaging at the Martinos Center for Biomedical Imaging).

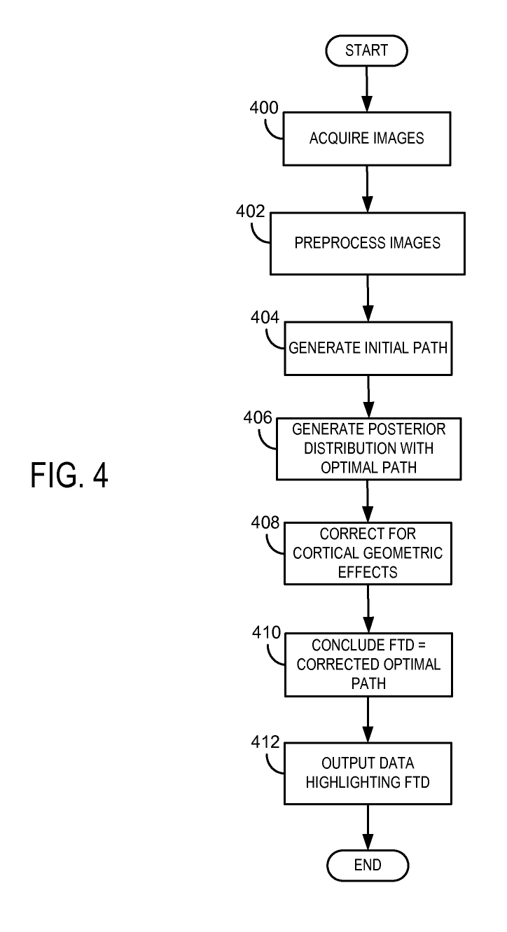

In light of the above, an example of steps for a method of automated detection of FTDs using MRI is illustrated in FIG. 4. This method includes acquiring images (process block 400). The images are then preprocessed or analyzed (process block 402) to obtain characteristics, such as cortical thickness, to create surface models. The processing or analysis at process block 402 may also obtain characteristics, such as ventricular labels, to select end points or anchors of probable FTDs, for example based on the surface models and ventricular labels. The processing or analysis at process block 402 may also obtain characteristics to align images to the surface models, to label white and gray matter, and to estimate intensity distributions of various tissue compartments in the images.

Following preprocessing, an initial transmantle path is generated (process block 404), for example, using a Catmull Rom spline representation with the selected end points. Based on the initial path, a posterior distribution may be generated (for example, using the Markov-Chain Monte-Carlo method), including an desired or optimal path, as well as additional, less likely paths (process block 406). A correction technique may then be applied to remove cortical geometric effects from the posterior distribution, further defining a corrected optimal path (process block 408). This corrected desired or optimal path can then be concluded as being an FTD (process block 410). Output data is reported outlining the FTD (process block 412), for example, by displaying an inflated cortical surface model highlighting the FTD. These process steps can be used with the MRI system 100 of FIG. 1 described above, or another imaging system. The above process steps of the method are further discussed in the following.

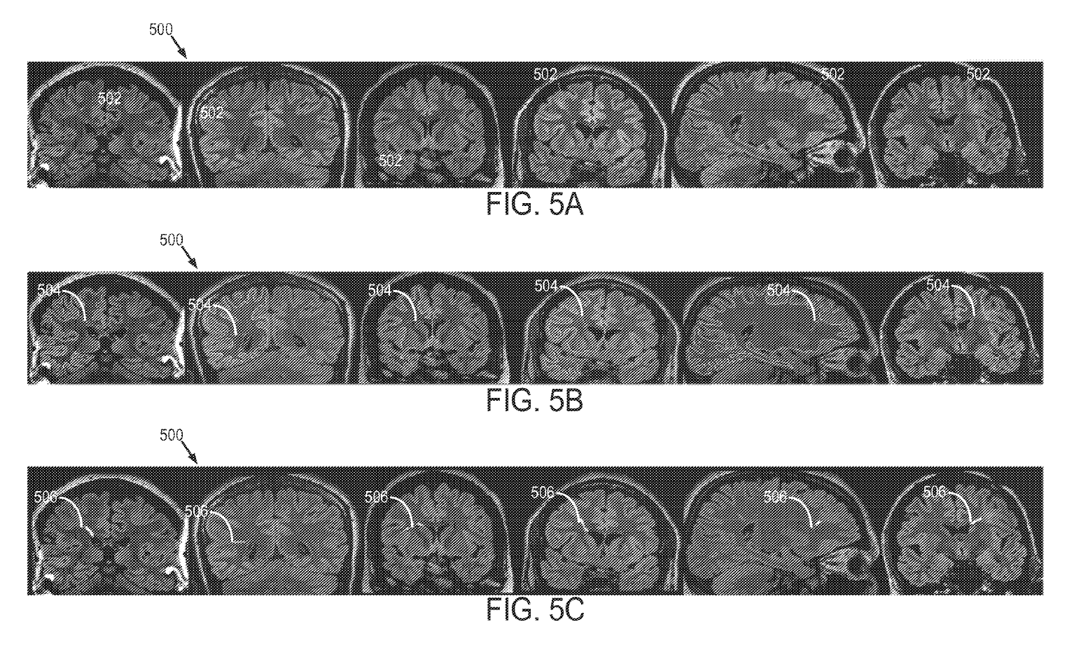

With respect to imaging (that is, the data acquisition process block 400 above), whole brain volumes can be collected, for example, following institution protocols for clinical epilepsy. In some examples, suitable imaging data can be acquired from 1 mm isotropic T1-weighted scans, including FLASH or motion-corrected multi-echo MPRAGE, 1 mm isotropic T2-SPACE FLAIR scans, or other suitable imaging techniques. An example of the appearance of FTDs is illustrated in FIG. 5A, which shows an inversion-prepared T2-SPACE FLAIR image 500 with the location of the FTD indicated by an arrow 502, as further discussed below.

Example features for accurate, sensitive, and specific localization of FTDs is now described. The features most typically used in detecting the presence of Type II FCDs are cortical thickness and FLAIR intensity. However, the defining characteristic of an FTD, the narrow band of abnormal intensities extending from the cortex to the ventricles, is not a local one, and hence is difficult or impossible to extract from local measures such as thickness of FLAIR intensity. For this reason, the present invention can explicitly model the entire path, then derive summary features from the path models to localize the FTDs.

A basic approach to path following, in which one starts in the cortex and steps from voxel to voxel searching for abnormally bright image intensities, may be inadequate for a number of reasons. The first reason is that such an approach tends to diverge into the brighter gray matter. This can be avoided using anatomical models of the cortex and subcortical structures, but will still be inadequate due to the small size of the FTDs (for example, with tails only one or two voxels wide) and the noisy nature of the underlying images. Once this type of local tracking takes an incorrect step, it will tend to depart dramatically from the true FTD.

Instead, the present invention also provides for a more global model that anchors ends of the path in the cortex and ventricles and a probabilistic technique that allows and accounts for noise in the images. A recently developed algorithm in the field of MRI tractography (Jbabdi, S., et al., 2007, which is incorporated herein by reference in its entirety), which follows such principles, can thus be adapted and modified for modeling of transmantle paths. Specifically, the present invention can model the expected characteristics of an FTD and neuronal migration paths by adhering, for example, to the following rules: (1) the modeled paths should follow abnormally bright FLAIR image intensities; (2) the modeled paths should be smooth; (3) the modeled paths should be close to minimal length (this is related to item 2); and (4) the modeled paths should traverse deep white matter and not approach the subcortical junction except near the cortical anchor. These rules may be prioritized or weighted differently in different implementations. The Catmull Rom spline representation satisfies these constraints and has a number of advantages, including the following: (1) the path is defined by a handful of control points, making the numerical minimization needed to estimate a likely path tractable; (2) the control points of Catmull Rom splines are guaranteed to lie on the path; and (3) the low-dimensional nature of the spline naturally imposes smoothness constraints on the paths. The most probable spline as well as the posterior distribution of all likely splines can then be computed using a Markov-Chain Monte-Carlo (MCMC) algorithm, as further discussed below.

According to the present invention, preprocessing (for example, at process block 402) may be completed using the FreeSurfer suite of tools for neuroanatomical analysis, which is an open source package designed for the automated analysis of brain MRI data. Of course, other tools may also be used. Briefly, the FreeSurfer suite of tools includes calculation of an affine Talairach transform, intensity normalization to remove bias fields induced by nonuniform receive coil sensitivities, removal of nonbrain tissue, whole-brain segmentation of cortical, subcortical, white-matter and ventricular structures, cortical segmentation, surface generation, topology correction, geometry-based atlas registration of cortical folding patterns, cortical parcellation and thickness calculation. The outputs of this processing stream that are most relevant for the detection and localization of FTDs are the surface models, which serve as anchors for one end of the transmantle path models, the ventricular labels, which anchor the other end, and the thickness, which is frequently abnormally large in subjects with FCDs. A boundary-based registration tool (such as that described by Greve, D & Fischl, B., 2009, which is incorporated herein by reference) may be used to robustly and accurately align the high resolution FLAIR images to the surface models, and whole-brain segmentation labeling of the white and gray matter is used to estimate the intensity distributions of various tissue compartments in the FLAIR images.

Regardless of the tools used, the following energy functional can be used to describe how far any given spline departs from how a transmantle dysplasia path should appear (in accordance with the desired properties described above):

E(Pᵢ)=λ_{I}I(Pᵢ)+λ_{L}L(Pᵢ)+λ_{S}S(Pᵢ)+λ_{V}V(Pᵢ), Eqn. (3);

where I(Pᵢ) is the intensity penalty for the path P anchored at the ith vertex in the surface, V(Pᵢ) counts the number of voxels that are not labeled white matter to encourage the splines to avoid (for example, the basal ganglia), L(Pᵢ) is the length penalty, S(Pᵢ) is the penalty for approaching the gray/white surface too closely (that is, it encourages the splines to stay in the interior of the white matter), and the λ coefficients define the relative weight assigned to each term. For the intensity term I(Pᵢ), the distribution of FLAIR intensities is modeled using a Gaussian distribution, and the gray and white matter class means and variances are estimated using the whole-brain segmentation of the registered T1-weighted image. I(Pᵢ) then encourages the paths to traverse voxels with intensities that are in the normal gray matter range (accordingly, this term amounts to a log-likelihood of the image appearance along the path assuming spatial independence in the imaging noise, and a Gaussian noise model).

The length penalty L(Pᵢ) may be given by the length of the path in millimeters (“mm”), thus discouraging paths that are too tortuous. Finally, for the surface interior penalty S(Pᵢ) a thresholded linear penalty may be chosen that does not affect paths that are in the interior at all, but penalizes those that approach the surface too closely, for example:

$$S ( {P}_{i} ) = {\int }_{{P}_{i}}^{ } H ( D ( x ) - {D}_{max} ) d x , H ( x ) = \{ \begin{matrix}x , x > 0 \\ 0 , otherwise\end{matrix} , \qquad Eqn . ( 4 ) ;$$

where Dₘₐₓ represents the closest that the path is allowed to approach the gray/white junction without incurring any penalty (for example, set to 2.5 mm), and D(x) gives the distance of location x in the volume to the closest point on the gray/white surface model. This term prevents paths from “hugging” the gray/white boundary, which would otherwise be a viable solution due to partial volume effects creating brighter appearing voxels at the subcortical junction.

With respect to path initialization (at process block 404), it may be desired to generate an initial path that can be deformed to minimize Equation 3 above. For this purpose a binary segmentation of the lateral ventricles is generated and from it a constrained distance transform is created, where the distances are constrained to be in the interior of the white matter. The spline is then initialized for each point in the cortex by numerically integrating the negative of the gradient of the distance transform. That is, the path starts in the cortex and follows decreasing distance transform values until it reaches the ventricles. This amounts to a minimal interior path from the point in the cortex to the lateral ventricles. For a small number of points there are local minima in the distance transform that prevent this procedure from reaching the ventricles. For such cases, the distance transform can be spatially smoothed before recomputing the gradient until a path reaching the ventricles can be found. This may result in paths that leave the interior of the white matter. This is not a concern, however, as the energy functional defined in Equation 3 encourages such paths to quickly return to the white matter during numerical minimization.

Due to the presence of noise in the images as well as the large size of the space of possible splines connecting the cortex with the lateral ventricles, it would be desirable to acquire an estimate of both the most likely spline under Equation 3, but also critically some measure of the uncertainty associated with that spline. A natural probabilistic tool to use in this case is the Markov-Chain Monte-Carlo (MCMC) method, which is designed to allow the exploration of the posterior distribution of otherwise intractable probabilistic spaces. Recently, MCMC techniques have been used in the MRI tractography to estimate the probability of a connection existing between disparate parts of the brain. The present invention employs an analogous use of MCMC techniques: that of constructing both the most likely spline at each point in the cortex, as well as an estimate of the spatial uncertainty in the distribution of likely splines (at process block 406).

MCMC is a reasonably powerful procedure that allows the construction of the high probability portion of a posterior distribution with relatively few assumptions. The basic idea of MCMC is to start with some estimate, in this case an initial path as described above, then perturb the path and evaluate the energy of the new path. The perturbation of the path is accomplished by drawing a sample from a “jumping” or “proposal” distribution, then moving a randomly selected control point by this amount. If the energy has decreased (that is, the path is more probable), the sample is accepted. If the energy has increased, then the path is accepted with a small probability; otherwise it is rejected and a new sample path is drawn. Running this algorithm for tens of thousands of samples converges to an estimate of the posterior distribution after a “burn-in” period of a specific number of iterations. Because the samples in MCMC are correlated, a “jumping width” must also be defined that specifies the correlation length of the samples (that is, how many samples must be skipped to before the new sample is uncorrelated with the previous one).

In one specific example, MCMC can be executed using a Gaussian proposal distribution with a 5 mm standard deviation, a 1000 iteration burn-in period and a jumping width of 5. Acceptance of an energy increase is randomly decided using an exponential distribution with a dispersion of 0.5. That is, the energy of the previous sample is subtracted from that of the potential new samples, divided by 0.5 and exponentiated. A uniform random number in [0,1] is then drawn, and if this number is below the exponential value computed above, the new sample is retained. This allows small energy increases to be accepted at a high rate, while making large positive energy changes unlikely to be accepted, preventing for example, the splines from leaving the interior of the white matter. The splines can be defined using five control points, and the coefficients may be set to λ_{I}=1, λ_{V}=200, λ_{L}=5, and λ_{S}=1000.

The MCMC algorithm can, therefore, be used to construct the most probable path from each point in the cortex to the ventricular system, as well as the total posterior probability of a path integrated across the cortex. The total posterior probability is accomplished by counting how often a path in the MCMC algorithm passes through every voxel. The total number of paths passing through a voxel is then a sensitive measure of how likely that voxel is to be a member of a transmantle path.

The paths modeled using the MCMC algorithm can provide a wealth of information that is potentially predictive of the existence and location of a transmantle dysplasia. One challenge in localizing the paths is distinguishing true heterotopias from other abnormally bright regions in the white matter such as Virchow-Robin spaces, leukoaraiosis and other non-specific foci of increased T2 signal. The defining characteristic of the transmantle dysplasias is their path-like appearance. That is, they are narrow “tubes” of bright T2/FLAIR intensities, as opposed to other causes of abnormal intensities that will increase the log likelihood but are not conical in appearance. The narrow nature of the transmantle dysplasias implies that the posterior distribution of the paths generated by the MCMC algorithm should be tight in true FTDs without much spatial spread, whereas in other sources of T2-brightening in the white matter there will be many possible paths that go through the bright regions, resulting in a spreading of the posterior distribution.

One problem with examining the posterior distribution for this signature of the dysplasia is that it can confound cortical geometry with intensity abnormalities. For example, when displaying the posterior distribution of path probabilities on inflated surface maps or in the volume (for ease of interpretation by neuroradiologists), the posterior distribution will initially be high precisely in the tail of the FTD, as many paths pass through these bright-appearing regions on FLAIR images. That is, there are “bottlenecks” in the cortex, in which many paths must pass through a thin region of the white matter, yielding a high posterior distribution that is reflective of cortical geometry rather than tissue properties. This would cause false positives near narrow bottlenecks in the cortex, such as at the thin base of a large gyrus that gives rise to high probabilities even in normal appearing tissue. In order to correct for this effect (that is, this geometry-induced probability), the MCMC or other algorithm can be executed on a synthesized image in which the FLAIR intensities in the interior of the white matter are replaced with random samples drawn from an appropriate Gaussian distribution, including the same mean and standard deviation as healthy-appearing white matter. This generates a posterior distribution that is only reflective of cortical geometry, which can be then removed from the distribution generated using the true data, yielding a corrected posterior distribution in which the effects of cortical geometry have been removed. Thus, the correction procedure can disentangle the effects of geometry from those of tissue appearance, resulting in increased specificity for the corrected posterior distributions.

### Example

The above processing methods and techniques were applied in a study examining the feasibility of aspects of the present invention. The study included six patients with post-operative diagnosis of FCDs, but a “negative” diagnosis from conventional MRI procedures and examination. In the study, MRI images were analyzed in accordance with methods of the present invention described above, and all six FTDs previously missed on clinical reads were detected, with an average of more than 15 years of potentially treatable seizures. The results of the study therefore indicated that the methods of the present invention can help identify possible FTDs in cases in which the dysplasias would otherwise have gone undetected, resulting in decades of potentially treatable seizures. The following paragraphs further describe materials, methods, and results of the study.

The six subjects used in the study were identified as having surgery for FCDs that carried a post-operative diagnosis of FTD, or had seizure freedom for at least 6 months post-surgery. All subjects had lesions that were not initially identified on MRI, with a mean time between seizure onset and diagnoses of 15±9 years. The clinical summaries are listed in Table 1 below.

TABLE 1
CLINICAL SUMMARY OF SUBJECTS IN STUDY

| | | | Post-operative |
|---|---|---|---|
| | Patient Age | | Diagnosis/Seizure |
| Patient ID | for MRI | Epilepsy | Freedom |
| 1 | 15 F | Patient seizures | Right sensory- |
| | | since age 12 | motor strip, type Ia, |
| | | | seizure freedom 1 |
| | | | year. |
| 2 | 18 F | Patient seizures | Right parietal FTD |
| | | since age 2 years | resection with |
| | | | seizure freedom 1 |
| | | | year |
| 3 | 40 F | 10 years of | Right FTD with 2 |
| | | nocturnal seizures | years seizure |
| | | with 10 years of | freedom |
| | | normal MRI | |
| | | reports | |
| 4 | 39 M | 16 years of | Resected right |
| | | intractable | frontal FTD with 1 |
| | | epilepsy with | month seizure |
| | | normal EEG; | freedom |
| | | MEG showed | |
| | | right frontal | |
| | | discharges | |
| 5 | 16 F | Intractable | Resected Right |
| | | epilepsy since | Frontal FCD, with |
| | | age 4 | 18 months of |
| | | | seizure freedom |
| 6 | 38 F | Intractable | Subpial transections/ |
| | | epilepsy since | subcortical |
| | | age 6 | stimulator trial, |
| | | | with markedly |
| | | | reduced seizure |
| | | | frequency |

Results were generated from the six patients described above using methods of the present invention. In particular, FreeSurfer surfaces were reconstructed for each subject from a T1-weighted image. The FLAIR images were registered to the surfaces using boundary based registration for each of the six subjects, as shown in FIG. 5A (where the actual FTDs are shown in each scan 500 by arrows 502). Next, the paths were initialized in accordance with the path initialization techniques described above, with a 1 mm blurring kernel applied to the constrained ventricular distance transform. The MCMC algorithm was then used to construct the most probable path from each point in the cortex to the ventricular system. FIG. 5B illustrates the most probable path 504 constructed using this procedure in the FTD. As shown in FIG. 5B, the most probable path in each subject accurately tracks the region of FLAIR hyper-intensity that is characteristic of transmantle dysplasias. Finally, FIG. 5C shows the total posterior probability 506 of a path integrated across the cortex.

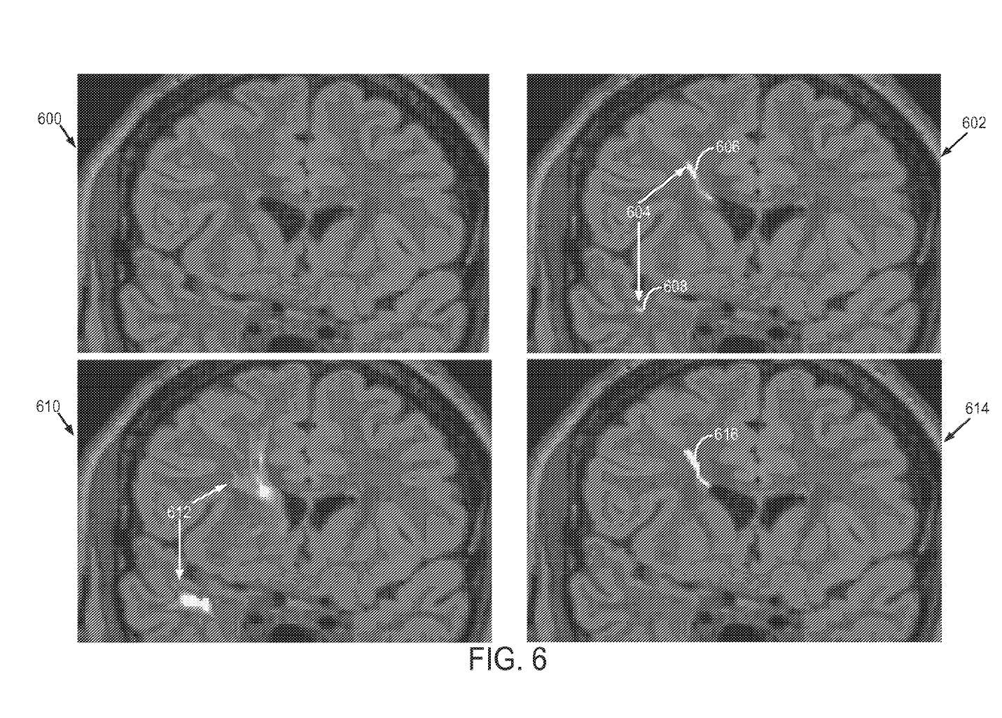

FIG. 6 illustrates a specific example of the correction procedure (also considered a normalization procedure) for removing the effects of cortical geometry, as described above, carried out on data from one of the subjects in the study (in particular, an 18-year-old patient with intractable epilepsy when presenting for advance neuroimaging evaluation). The top left image 600 is a T2-SPACE FLAIR showing the location of the subtle right-hemisphere transmantle dysplasia that is only visible at 1 mm isotropic or higher resolution. The top right image 602 is the posterior probability 604 of each point being in a transmantle dysplasia integrated over the entire right hemisphere. As shown, this top right image 602 properly highlights the dysplasia 606 but contains false positives 609, particularly in the temporal lobe at the base of narrow strands where cortical geometry necessitates the passage of many paths. The bottom left-hand image 610 shows the posterior probability 612 when the input image intensities are randomized, disentangling the effects of geometry from tissue properties. Subtracting this image 610 from the top right image 602 yields the image at the bottom right 614, which has been normalized for the effects of geometry, perfectly highlighting the transmantle dysplasia 614.

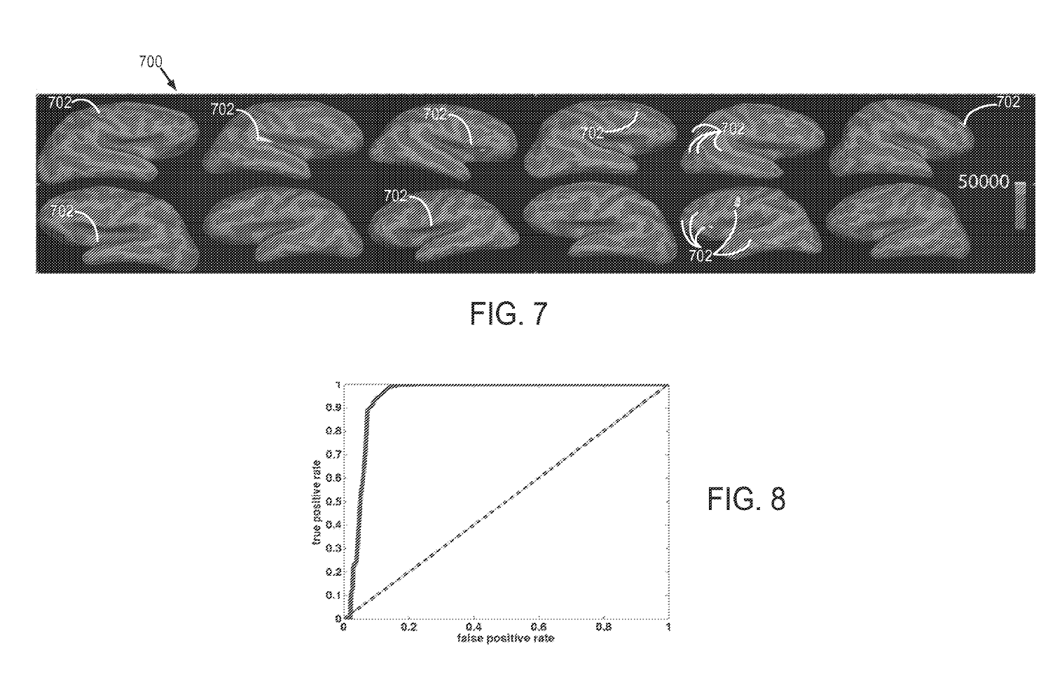

This correction procedure was applied to the data from all six subjects in the study and, as shown in FIG. 7, the results were sampled onto inflated cortical surface models 700 so that all of the lateral cortex, including FTDs 702, could be seen in a single view. More specifically, the posterior distribution was computed across all points in each hemisphere, and then for the optimal spline at each point over a segment of the spline approximately 3-5 millimeters interior to the cortical surface. This avoids features such as the occipital horn of the lateral ventricles and other deep white matter regions that can appear bright on T2/FLAIR images. The results of the study, as shown in FIG. 7, illustrate that every dysplasia was correctly marked using a single threshold, with only a handful of false positives.

A receiver operating characteristic (“ROC”) analysis was completed for the six subjects in the study by computing the true and false positive rates over lateral neocortical regions of the six affected hemispheres, using manual labelings of the FTDs drawn by a neuroradiologist. This was carried out by varying the threshold on the spline posterior shown in FIG. 7. For each threshold, the number of vertices that were labeled as dysplasia that were in the manual label (true positive), in the manual label but not above threshold (false negative), not in the manual label and above threshold (false positive) and not in the manual label and below threshold (true negative) over the entire range of values in the spline posteriors were computed. These were then used to compute the true positive and false positive rates, plotted against each other in a standard ROC curve in FIG. 8. Numerically integrating the ROC curve yielded an area under the curve (“AUC”) of 0.945, and a specificity of 0.9 at a sensitivity of 0.95, showing the excellent detection performance of the algorithm.

Thus, the results of the above-described feasibility study illustrate that the present invention has a high sensitivity and acceptable specificity, validating it as a screening tool for these difficult-to-detect cortical abnormalities. This aspect of the present invention can therefore help clinicians diagnose FTD in cases in which the dysplasias would otherwise have gone undetected, preventing years or decades of potentially treatable seizures in these patients and avoiding the concomitant neurologic damage associated with chronic seizures.

The present invention has been described in terms of one or more preferred embodiments, and it should be appreciated that many equivalents, alternatives, variations, and modifications, aside from those expressly stated, are possible and within the scope of the invention.

1. A magnetic resonance imaging (MRI) system, comprising:
a magnet system configured to generate a polarizing magnetic field about at least a portion of a subject arranged in the MRI system;
a magnetic gradient system including a plurality of magnetic gradient coils configured to apply at least one magnetic gradient field to the polarizing magnetic field;
a radio frequency (RF) system configured to apply an RF field to the subject and to receive magnetic resonance signals therefrom in parallel; and
a computer system programmed to:
control operation of the magnetic gradient system and RF system to acquire image data of a subject brain at a first resolution;
analyze the acquired image data to determine a thickness of cerebral gray matter;
match a left cerebral hemisphere to a right cerebral hemisphere based on corresponding geometric features of the left cerebral hemisphere and the right cerebral hemisphere;
generate a difference map comparing corresponding thicknesses of the left cerebral hemisphere and the right cerebral hemisphere;
identify regions of abnormal differences in thickness on the difference map as potential regions containing focal cortical dysplasias;
generate images of the regions of abnormal differences in thickness from the acquired image data; and
display the images.

2. The system of claim 1 wherein the acquired image data is at a first resolution and the computer system is programmed to acquire additional image data of the regions of abnormal differences in thickness at a second resolution and to generate the images of the regions of abnormal differences in thickness from the additional image data.

3. The system of claim 1 wherein the second resolution is higher than the first resolution

4. The system of claim 3 wherein the second resolution is a voxel resolution of about 1 millimeter.

5. The system of claim 1 wherein the geometric features include sulci and gyri.

6. The system of claim 1 wherein the computer system is programmed to acquire additional image data of the regions of abnormal differences in thickness in one of the left cerebral hemisphere and the right cerebral hemisphere if the abnormal difference in thickness is positive and to acquire image data of the regions of abnormal differences in thickness in the other of the left cerebral hemisphere and the right cerebral hemisphere if the abnormal difference in thickness is negative.

7. The system of claim 1 wherein the abnormal differences in thickness include differences greater than or equal to about three millimeters.

8. The system of claim 1 wherein the computer system is further programmed to display the difference map highlighting the regions of abnormal differences in thickness.

9. The system of claim 1 wherein the computer system is programmed to determine the thickness of the cerebral gray matter by building models of a gray-white boundary and a pial surface of the cerebral gray matter based on the acquired image data.

10. A method for automatic detection of potential focal cortical dysplasias (FCDs) from medical images acquired using a medical imaging system, the method comprising:
acquiring, with the medical imaging system, image data of a subject brain at a first resolution;
analyzing the acquired image data to determine a thickness of cerebral gray matter;
matching a left cerebral hemisphere to a right cerebral hemisphere based on corresponding geometric features of the left cerebral hemisphere and the right cerebral hemisphere;
generating a difference map comparing corresponding thicknesses of the left cerebral hemisphere and the right cerebral hemisphere;
determining regions of abnormal differences in thickness on the difference map as potential regions containing focal cortical dysplasias;
acquiring additional image data of the regions of abnormal differences in thickness at a second resolution;
generating images of the regions of abnormal differences in thickness from the additional image data; and
displaying the images.

11. A system comprising:
a computer system programmed to:
access image data of a subject brain;
analyze the acquired image data to estimate signal intensity distributions of the acquired image data relative to compartments of the subject brain and to determine at least two anchor points of a potential transmantle path;
generate an initial transmantle path between the two anchor points,
determine a posterior distribution including an optimal transmantle path and additional transmantle paths based on the initial transmantle path;
apply a correction technique to remove cortical geometric effects from the posterior distribution;
determine a corrected optimal transmantle path from the corrected posterior distribution as a focal transmantle dysplasia; and
display an image highlighting the focal transmantle dysplasia.

12. The system of claim 11 wherein computer system is programmed to generate the initial transmantle path based on a Catmull Rom spline representation.

13. The system of claim 11 wherein computer system is programmed to generate the initial transmantle path to substantially follow abnormally bright signal intensities of the acquired image data.

14. The system of claim 11 wherein computer system is programmed to control a magnetic gradient system and a radio frequency (RF) system of a magnetic resonance imaging (MRI) system to acquire the image data using one of a magnetization prepared rapid gradient echo pulse sequence and fluid attenuated inversion recovery pulse sequence.

15. The system of claim 11 wherein the computer system is programmed to analyze the acquired image data to determine at least the two anchor points based on observed abnormal intensities within the acquired image data.

16. The system of claim 11 wherein computer system is programmed to select a first of the two anchor points to correspond to a location at the brain cortex and a second of the two anchor points to correspond to a location at the brain ventricles.

17. The system of claim 11 wherein the computer system is programmed to determine the posterior distribution using a Markov-Chain Monte-Carlo method.

18. The system of claim 11 wherein the computer system is programmed to perform the correction technique to include subtracting a synthesized posterior distribution based on cortical geometric effects from the posterior distribution.

19. The system of claim 11 wherein the displayed image is an inflated surface map of the subject brain.

20. A method for automatic detection of a focal transmantle dysplasia (FTD) comprising:
acquiring, using a medical imaging system, image data of a subject brain;
analyzing the acquired image data to determine at least two anchor points of a potential transmantle path;
generating an initial transmantle path between the two anchor points;
determining a posterior distribution including an optimal transmantle path and additional transmantle paths based on the initial transmantle path;
applying a correction technique to remove cortical geometric effects from the posterior distribution;
concluding a corrected optimal transmantle path from the corrected posterior distribution as the focal transmantle dysplasia; and
displaying an image highlighting the focal transmantle dysplasia.
